(19) United States
(12) Patent Application Publication
(10) Pub. No.: US 2012/0220580 A1
(43) Pub. Date: Aug. 30, 2012

(54) PREPARATION OF CAROTENOID DERIVATIVES AND THEIR APPLICATIONS

(75) Inventors: YANMEI LI, BEIJING (CN); QINGHUA LIU, BEIJING (CN)

(73) Assignee: BEIJING GINGKO GROUP BIOLOGICAL TECHNOLOGY CO., LTD., BEIJING (CN)

(21) Appl. No.: 13/450,565

(22) Filed: Apr. 19, 2012

Related U.S. Application Data
(63) Continuation-in-part of application No. PCT/CN2010/001478, filed on Sep. 25, 2010.

(30) Foreign Application Priority Data
Oct. 20, 2009 (CN) 200910236362.X

(51) Int. Cl. A61K 31/5377; A61K 31/336; A61P 3/04; C07D 407/12; A61K 31/341; C07D 413/12; C07D 303/40; C07D 301/00

(52) U.S. Cl. 514/232.5; 549/546; 514/475; 549/539; 549/315; 514/473; 544/111

(57) ABSTRACT

Compounds having the structure of general formula I or pharmaceutically acceptable salts thereof which are derivatives of fucoxanthin and fucoxanthol are disclosed. A preparation method of the compound and its use in weight reducing are also disclosed.

### CROSS-REFERENCES TO RELATED APPLICATIONS

The present application is a continuation-in-part application to International Application No. PCT/CN2010/001478, with an International Filing Date of Sep. 25, 2010, which claims the benefit of Chinese Patent Application No. 200910236362.X filed in the Chinese Intellectual Property Office on Oct. 20, 2009, the entire contents of which are incorporated herein by reference,

### 1. TECHNICAL FIELDS

This invention refers to the carotenoid derivatives, especially the derivatives of fucoxanthin and fucoxanthol type compounds, as well as their preparation. Further, it refers to the applications of these compounds in weight-reducing.

### 2. BACKGROUND OF THE TECHNIQUE

Carotenoids have long been thought as a type of pigments, however, their function of coloring up is only one of theirs, more of their applications are those embody their biological functions. There are many research literatures in this aspect, mainly on topics such as their uses as provitamin A, in cleaning up free radicals, in prevention of cancer and antitumors, in eye care and so on. In a sense, carotenoids are vitaminoids with vitamin functions.

As there are at least 9 double bonds in the molecules of carotenoids, they are very labile, easy to degrade under the effects of light, oxygen, moisture, heavy metals, oxidants or reductants; moreover, when they undergo changes of condition they are apt to change into isomers with lower activity, this justifies the necessity to transform them into preparations, which will greatly increase their adaptation to the environment and their stability. Besides, owing to the liposolubility of carotenoids, their direct applications in food and beverages are seriously restricted. Currently, they are not directly used in crystal form in food, feedstuff and in drugs as their assimilative rates are low, besides they have almost no coloring effect. Therefore, it is necessary to transform carotenoids into preparations to change their solubility, thereby increasing their assimilative rates as well as their coloring effects, and finally, broadening the ranges of their applications.

On the other hand, there are many factors affecting the bioavailability of carotenoids, which might affect the assimilation of carotenoids. such as the structure of carotenoids, their physical combinative status in food, contents of fat and protein in meals, change of pH values in the bodies of animals, concentrations of cholic acids and cholates in bile, content of pancreatin in intestinal tract, as well as in vivo vitamin A nutritional status, those are all the factors which might affect the transformation of carotenoids, or directly affect the activity of carotenoid dioxygenase, some inhibitors will also depress the assimilation of carotenoids.

Fucoxanthin, [3′-(Acetyloxy)-6′,7′-didehydro-5,6-epoxy-5,5′,6,6′,7,8-hexahydro-3,5′-dihydroxy-8-oxo-β,β-carotene], also known as pheophytin or marjoram algae flavin. They are mahogany materials, are one of the important members of carotenoid family. They are pigments contained in brown algae diatoms, golden algae and yellow-green algae. As components of photochemical system II, they participate in photosynthesis. After isolation, mahogany crystals may be obtained. They are xanthophyll type compounds, and are the materials that endow the brown algae with the brown color, therefore they can be labeled as the characteristic pigment of brown algae category, although sometimes they are also found in diatomeae and other algae. Their molecular formula is C₄₂H₅₈O₆, the structure formula is shown as below:

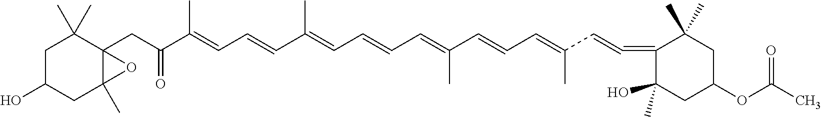

Fucoxanthin has a number of medicinal efficacies: It has strong antitumor effects and rather strong antioxidation characteristics, therefore it is an ideal dietary supplement; it has some efficacy in treating diabetes; and it has significant effect in weight-reducing. Researchers in Hokaido University, Japan, have proved that, fucoxanthin—a pigment contained in brown algae-would be a miraculous medicine for reducing body weight. Through experimenting with lab mice, the researchers found that by addition of food additives containing fucoxanthin at regular intervals, weight of mice decreased by 5˜10%. Altogether the scientists studied on more than 200 lab animals, they found in the experiments that, fucoxanthin can be used to eliminate the accumulation of fat in two ways: one is to activate a protein known as UCP1, which can promote the decomposition of fat; simultaneously it can stimulate the liver to generate DHA which have the function of reducing cholesterol level. Meanwhile, the scientists verified that use of fucoxanthin as an additive in condensed food would not induce the addiction of the experimental animals, or had any other side effects.

Fucoxanthol is a biological active constituent extracted from ascidian (sea squirt). The structural feature is that the 3′-acetyl group its molecule is changed into hydroxy. Its weight-reducing activity is much stronger than fucoxanthin (CA2609454A1).

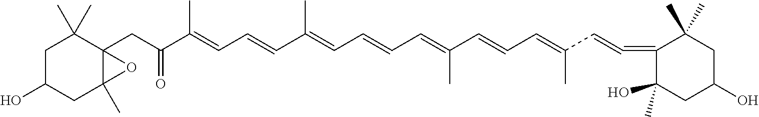

### CONTENTS OF THIS INVENTION

High lipophilicity of carotenoid compounds restricts their bioavailability, which in its turn also restricts appropriate evaluation of their biological effects. To solve this problem, this invention aims at modifying the structure of fucoxanthin and fucoxanthol, thereby improving their hydro-solubility and liposolubility, further, to change their assimilation in vivo, then to improve their bioavailability.

To attain this aim, the technical scheme adopted by this invention is to provide a series of fucoxanthin derivatives, to synthesize new fucoxanthin or fucoxanthol derivatives having prodrug functions (i.e., the medicinal activity of the derivative form). The new, bio-effect correlated fucoxanthin or fucoxanthol derivatives conjugated with precursor moieties, render far greater activity than fucoxanthin or fucoxanthol themselves, in particular, result in prominent weight-reducing effect.

This invention provides a type of compounds with general formula I, or their pharmaceutically acceptable salts:

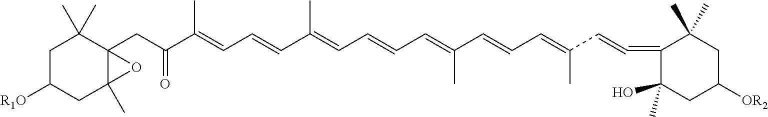

wherein said R₁ or R₂ is individually hydrogen, acetyl, citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, morpholinocarbamoyl, mannitol carbonyl, hexadecanoyl, linoleyl, linolenyl, arachidonyl, but both groups are not hydrogen simultaneously; or

R₁ or R₂ is groups independently forming ether groups with the individual oxygen they conjugate; or

R₁ or R₂ is citryl or succinyl individually being further esterified independently;

wherein, when R₁ is citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, hexadecanoyl, linoleyl, linolenyl, arachidonyl, or the group forming ether with the oxygen it conjugate, or the citryl or succinyl being further esterified, preferentially, said R₂ is acetyl;

wherein said “group forming ethereal group with the oxygen they conjugate”, is for instance C₁₋₆alkyl, C₆₋₁₂ aryl, aryl C₁₋₄alkyl, C₁₋₉heteroaryl or C₁₋₄ alkylheteroaryl and so on, more preferentially it is selected from C₁₋₄alkyl aryl, more preferentially it is benzyl;

wherein said “citryl or succinyl being further esterified”, means a di- or poly-carboxylic acids such as citric acid and succinic acid, wherein one of the carboxyl groups esterifies with one hydroxyl group in fucoxanthin or fucoxanthol, whereas at least one of the carboxyl groups of the acid further esterifies with an alcohol. For example, the product can be of the following structure:

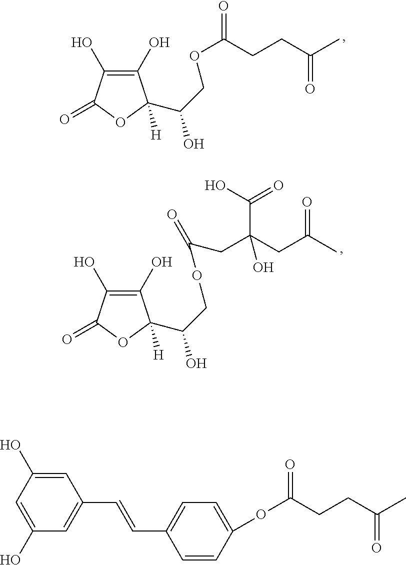

said “pharmaceutically acceptable salts” are the sodium or potassium salts formed by the acids with the alkaline metals.

In the embodiments of this invention, R₁ is citryl or its acyl with a sodium ion, succinyl or its acyl with a sodium ion, or 1-substituted-ascorbic acid succinyl, 1-substituted-ascorbic acid citryl or its acyl with a sodium ion. Said “acyl with a sodium ion” or the “acyl sodium salts” as stated in the Claims is a di- or poly-carboxylic acids such as citric acid and succinic acid, wherein one of the carboxyl groups esterified with one hydroxyl group in fucoxanthin or fucoxanthol, whereas at least one of the carboxyl (or hydroxyl) group of the acid conjugate with sodium ion to form salt, whereas R₂ is acetyl.

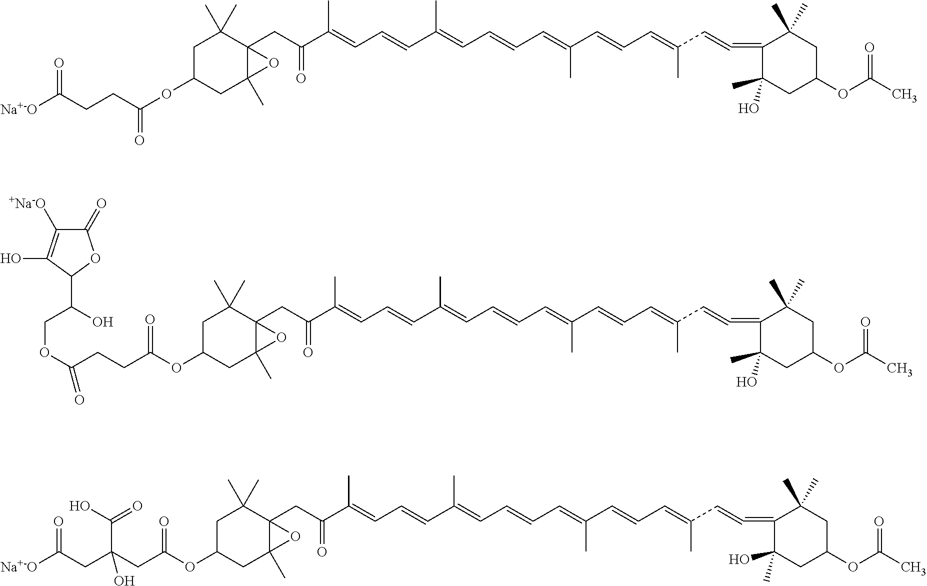

The purpose to conjugate an R₁ side chain in the 3-position of fucoxanthin molecule, and to conjugate R₁, R₂ side chains in the 3,3′-position of fucoxanthol molecule is to improve their bioavailability, increase their absorption in the intestinal tract or their solubility in injections.

In this invention, a drug composition includes a kind of pharmaceutically acceptable adjuvant or diluent, as well as a compound with the structure as shown in general structure I, are provided.

In this invention, the preparation methods of said general formula I compounds are also provided. Due to the contents of olefinic bonds, epoxide groups and keto carbonyl groups in the structures of fucoxanthin and fucoxanthol, during the esterification of the hydroxyl groups, strong alkalines cannot be used, otherwise the epoxide groups will be hydrolyzed. It is known to the technicians in this field that, in this case, only non-nucleophilic alkalines such as pyridine and substituted pyridines can be used. Fucoxanthin and fucoxanthol are only stable under the low-temperature and dark conditions, otherwise inter-double bonds and inter-molecule polymerization will occur. During the reaction substituting the leaving group in the precursors, nucleophilic reagents should be used together with the de-protonized alcohol. The leaving groups can be (but are not restricted to) Cl, Br, toluene-p-sulfonyl, methylsulfonyl, p-bromophenylsulfonyl or trifluoromethylsulfonyl.

Following is a non-restrictive embodiment of the synthetic flow path of fucoxanthin- and fucoxanthol derivatives.

Using fucoxanthin as the raw material:

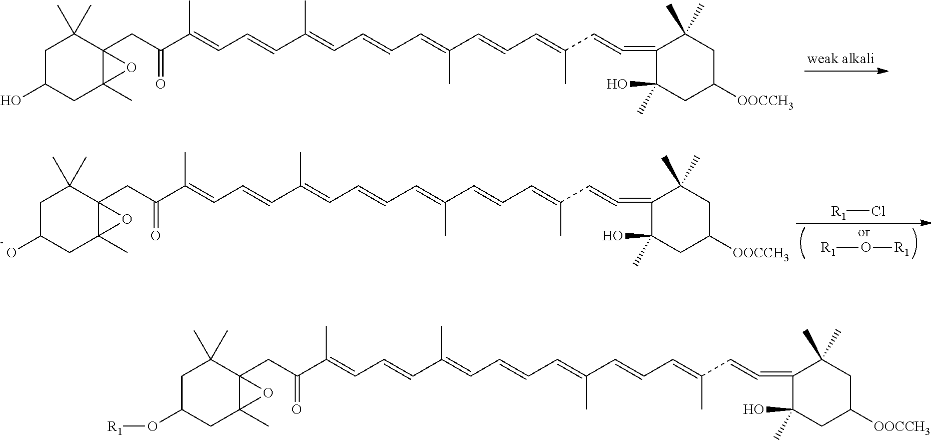

Using fucoxanthol as raw material

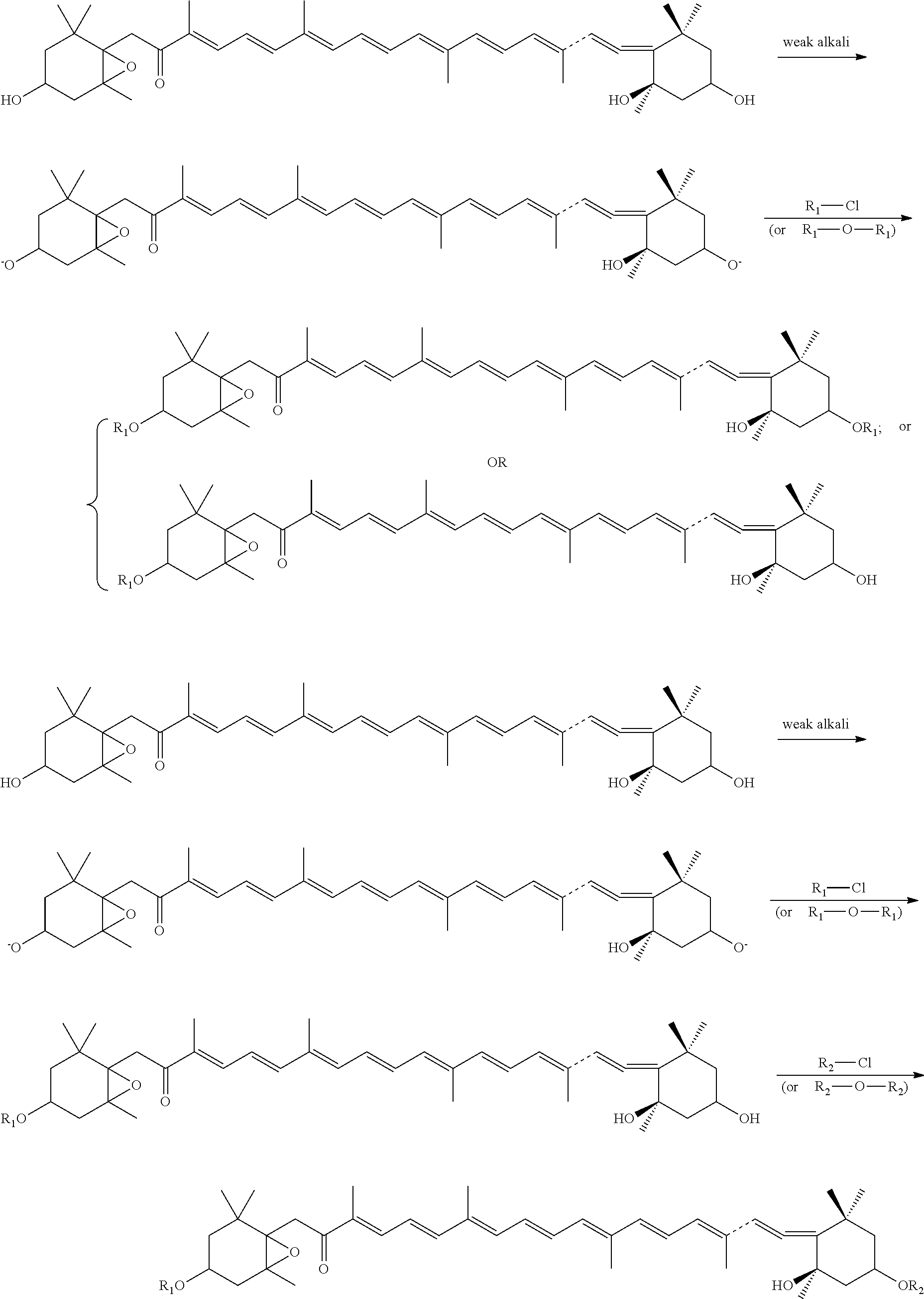

wherein R₁, or R₂ is individually hydrogen, acetyl, citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, morpholinocarbamoyl, mannitol carbonyl, hexadecanoyl, linoleyl, linolenyl, arachidonyl, but both groups are not hydrogen simultaneously; or

R₁, or R₂ is the group individually forming ether with the oxygen conjugating with them; or

R₁, or R₂ is citryl or succinyl, individually being further esterified; or

another esterification technique, the DCC (1,3-Dicyclohexylcarbodiimide) method was used, to esterify the alcoholic hydroxyl in the 3 position of fucoxanthin, or fucoxanthol; or one of the alcoholic hydroxyl group in 3 and 3′-position, or both, of fucoxanthin, whereas other groups remain stable, besides carboxylic acids can be used to conduct the reaction. The reaction is convenient and rapid.

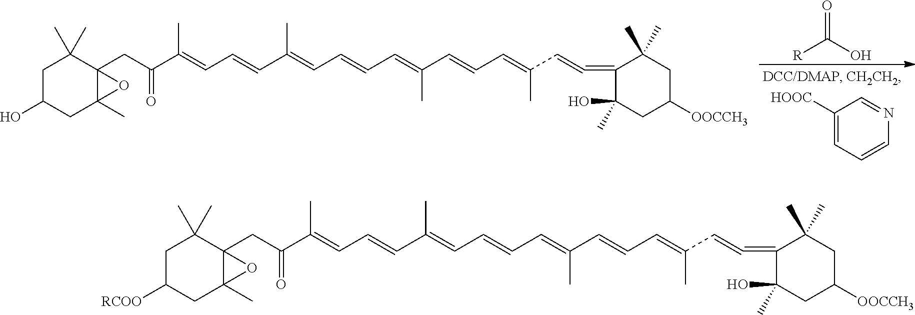

wherein R is individually alkyl, substituted-alkyl.

DCC esterification method uses 4-dimethylaminopyridine (DMAP) as the catalyst, DCC is used as dehydrating reagent.

Fucoxanthin is added into a dry flask with a magnetic stir bar. 1-3 times (molar) of carboxylic acid, 1-3 times (molar) of dehydrating reagent DCC, 0.2-3 times (molar) of catalyst 4-dimethylaminopyridine and enough amount of methylene chloride to dissolve all of the reagents are added into the flask, reaction is conducted under room temperature for 24-72 h with stirring. After the completion of the reaction the reactant is vacuum filtrated to remove unreacted DCC and the ureide the reaction produced. After removing the solvent methylene chloride, derivatives of fucoxanthin or fucoxanthol can be obtained by means of column chromatographic separation.

On the other hand, this invention further provides a weight reducing method, preferentially for mammalians, most preferentially for human being. This method comprises administration of the compounds or composition described in this invention to the animals. This invention features the treatment or prevention of the diseases correlated with obesity. The method to treat or to prevent obesity may include oral, topical, intravenous, intramuscular or subcutaneous administration of the compounds or composition described in this invention.

By means of structural modification to fucoxanthin and fucoxanthol, this invention improves the water solubility and liposolubility of fucoxanthin and fucoxanthol. Their solubility in the chylomicrons in intestinal tract, as well as their per os bioavailability, can be increased through esterification or etherification of the compounds, thereby leading to significant level of fucoxanthin, fucoxanthol or their derivatives in plasma and in solid organs. As a result of more significant weight reducing effect than the parent compounds fucoxanthin and fucoxanthol can be brought about. Synthetic analogs of fucoxanthin and fucoxanthol or their derivatives may improve the water solubility of these compounds during oral, topical, intravenous, intramuscular or subcutaneous administration, and greatly boost bioavailability for weight reducing.

The compounds described in this invention can reduce the body weight of the subjects, reduce fat content in the body, the tissue of fat pads around the testicles, and the weight of surrounding fat pads; moreover, these derivatives are brand-new compounds, they have more significant ability to reduce the body weight than fucoxanthin and fucoxanthol themselves, and show remarkable features and prominent improvements.

### EMBODIMENTS OF THIS INVENTION

Following are further demonstrations to this invention with embodiments. It should be understood that these embodiments are only used for demonstration, and are by no means a restriction to the protection scope of this invention.

All the reagents were from the commercial sources, they were not treated before use, the solvents were used directly in the reactions and separations, unless otherwise stated.

All the reactions were conducted in the atmosphere of inert gases, which included nitrogen and argon, and the reaction was held in the dark. Fucoxanthin and fucoxanthol were self-produced in the Beijing Gingko Group Biol. Tech. Co. Ltd. Thin layer chromatography (TLC) was conducted by Qingdao Haiyang GF254 silica gel plates. NMR spectrograms were recorded using a Bruker Advance NMR (500 MHz), and MS tests were used a ThermoFinnigan AQA MS.

The structures —OOCCH₃ or —OCOCH₃, —OOCR or —OCOR are respectively the abbreviations of

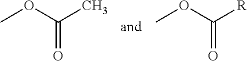

in fact.

### Example 1

### Synthesis of 1(fucoxanthin citrate)

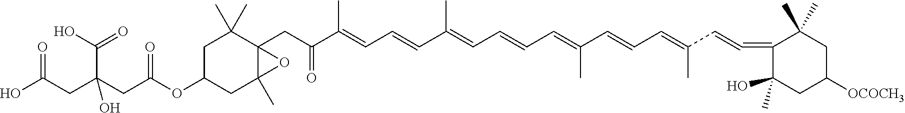

5 mmol fucoxanthin was dissolved in 100 ml methylene chloride at room temperature, 105 mmol N,N-Diisopropylethylamine, 50 mmol citric anhydride and 2.5 mmol 4-(dimethylamino)pyridine were added into the solution. The reaction mixture was stirred at room temperature for 40 h, the reaction mixture was then diluted with methylene chloride, the reaction was terminated with NaCl solution/0.8M hydrochloric acid (120 mL/25 mL) mixture. The reactant was extracted with methylene chloride, the organic layers were mixed, the mixed solution dried with anhydrous Na₂SO₄ and condensed. The product was separated by purified by silica gel column chromatography to obtain fucoxanthin monocitrate. No existence of fucoxanthin was confirmed by TLC and HPLC.

ESI m/z: 833.44 (M⁺+1); (Found: M⁺, 832.44 C₄₈H₆₄O₁₂).

¹HNMR Data (500 M Hz): δ_{H} 0.95 (3H, s, 1-Me^{eq}), 1.04 (3H, s, 1-Me^{ax}), 1.07 (3H, s, 1′-Me^{eq}), 1.22 (3H, s, 5-Me), ˜1.35 (2-H^{ax}), 1.35 (3H, s, 5′-Me), 1.39 (3H, s, 1′-Me^{ax}), 1.41 (1H, t, J 12, 2′-H^{ax}), ˜1.49 (2-H^{eq}), 1.51 (1H, t, J 13, 4′-H^{ax}), 1.79 (1H, dd, J 14 and 9, 4-H^{ax}), 1.82 (3 H, s, 9′-Me), 1.95 (3H, s, 9-Me), 1.99 (6H, s, 13-+13′-Me), ˜2.00 (2′-H^{eq}), 2.04 (3H, s, OAc), 2.29 (1H, ddd, J 13, 4 and 2, 4′-H^{eq}), 2.32 (1H, brdd, J 14 and 4.5, 4-H^{eq}), 2.60 and 3.66 (each 1H, d, J 18, 7-H₂), 2.73 and 2.48 (each 1H, d, J 18, 4″-H₂), 2.77 and 2.52 (each 1H, d, J 18, 2″-H₂), 5.30 (1H, m, 3-H), 5.38 (1H, m, 3′-H), 6.06 (1H, s, 8′-H), 6.13 (1H, dd-like, J 11 and 1, 10′-H), 6.27 (1H, br d, J 11.5, 14′-H), 6.35 (1H, d, J 15, 12′-H), 6.41 (1H, brd, J 11.5, 14-H), 6.57 (1H, dd, J 15 and 11, 11-H), 6.60 (1H, dd, J 15 and 11, 11′-H), 6.64 (1H, dd, J 14.5 and 11.5, 15-H), 6.67 (1H, d, J 15, 12-H), 6.75 (1H, dd, J 14.5 and 11.5, 15′-H) and 7.15 (1H, br d, J 11, 10-H).

### Example 2

### Synthesis of 2(fucoxanthin succinate)

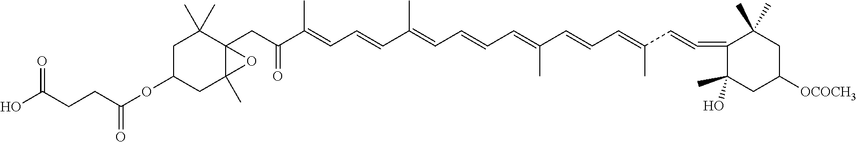

10 mmol fucoxanthin was dissolved in 230 ml methylene chloride at room temperature, the solution was added with a N,N-Diisopropylethylamine, 50 mmol succinic anhydride, and 6 mmol 4-(dimethyl amine)pyridine. The mixture was stirred at room temperature for 30-45 h, the reactant was subsequently diluted with the solvent methylene chloride, the reaction was terminated with NaCl solution/0.1M HCl (70 mL/9 mL), the reactant was then extracted with methylene chloride, the organic layers were mixed. The mixed solution was dried with a desiccant, and condensed to obtain solid material. No existence of fucoxanthin was confirmed by TLC and HPLC. The reactant mixture was separated by a silica chromatographic column to obtain fucoxanthin monosuccinate.

ESI m/z: 759.44 (M⁺+1); (Found: M⁺, 758.44, C₄₆H₆₂O₉).

¹HNMRData: δ_{H} (500 M Hz) 0.94 (3H, s, 1-Me^{eq}), 1.03 (3H, s, 1-Me^{ax}), 1.07 (3H, s, l′-Me^{eq}), 1.21 (3H, s, 5-Me), ˜1.34 (2-H^{ax}), 1.36 (3H, s, 5′-Me), 1.40 (3H, s, 1.42 (1H, t, J 12, 2′-H^{ax}), 1.49 (2-H^{eq}), 1.52 (1H, t, J 13, 4′-H^{ax}), 1.80 (1H, dd, J 14 and 9, 4-H^{ax}), 1.83 (3H, s, 9′-Me), 1.96 (3H, s, 9-Me), 1.99 (6H, s, 13-+13′-Me), ˜2.00 (2′-H^{eq}), 2.05 (3H, s, OAc), 2.30 (1H, ddd, J 13, 4 and 2, 4′-H^{eq}), 2.33 (1H, brdd, J 14 and 4.5, 4-H^{eq}), 2.61 and 3.67 (each 1H, d, J 18, 7-H₂), 2.62 (2H, dd, J 18, 3″-H₂), 2.53 (2H, dd, J 18, 2″-H₂), 5.31 (1

H, m, 3-H), 5.39 (1H, m, 3′-H), 6.07 (1H, s, 8′-H), 6.12 (1H, dd-like, J 11 and 1, 10′-H), 6.26 (1H, br d, J 11.5, 14′-H), 6.34 (1H, d, J 15, 12′-H), 6.40 (1H, brd, J 11.5, 14-H), 6.56 (1H, dd, J 15 and 11, 11-H), 6.61 (1H, dd, J 15 and 11, 11′-H), 6.60 (1H, dd, J 14.5 and 11.5, 15-H), 6.66 (1H, d, J 15, 12-H), 6.74 (1H, dd, J 14.5 and 11.5, 15′-H) and 7.14 (1H, br d, J 11, 10-H).

### Example 3

### Synthesis of 3(fucoxanthin succinate sodium salt)

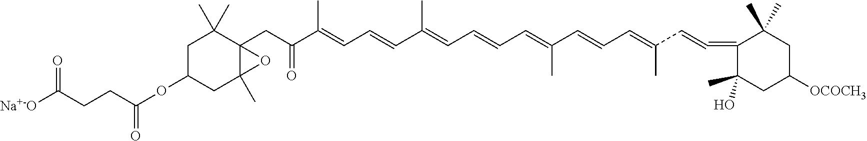

1 mmol fucoxanthin monosuccinate and 100 mL ethyl alcohol were stirred in a 250 mL round bottom flask in an inert gas atmosphere and at room temperature, 1 mmol solid sodium ethylate was added, stirred overnight. The precipitation was filtered out the next day, washed with small amount of ethyl alcohol and subsequently with chloroform or methylene chloride to obtain a solid. Fucoxanthin succinate sodium salt was obtained by column chromatography.

ESI m/z: 781.42 (M⁺+1); (Found: M⁺, 780.42, C₄₆H₆₁O₉Na).

¹H NMR Data: δ_{H} (500 M Hz) 0.95 (3H, s, 1-Me^{eq}), 1.02 (3H, s, 1-me), 1.08 (3 H, s, l′-Me^{eq}), 1.22 (3H, s, 5-Me), ˜1.34 (2-H^{ax}), 1.35 (3H, s, 5′-Me), 1.39 (3H, s, 1′-Me^{ax}), 1.43 (1H, t, J 12, 2′-H^{ax}), ˜1.47 (2-H^{eq}), 1.50 (1H, t, J 13, 4′-H^{ax}), 1.81 (1H, dd, J 14 and 9, 4-H^{ax}), 1.82 (3H, s, 9′-Me), 1.95 (3H, s, 9-Me), 1.98 (6H, s, 13-+13′-Me), ˜2.00 (2′-H^{eq}), 2.04 (3H, s, OAc), 2.29 (1H, ddd, J 13, 4 and 2, 4′-H^{eq}), 2.32 (1H, brdd, J 14 and 4.5, 4-H^{eq}), 2.60 and 3.66 (each 1H, d, J 18, 7-H₂), 2.78 (2H, dd, J 18, 3″-H₂), 2.59 (2H, dd, J 18, 2″-H₂), 5.31 (1H, m, 3-H), 5.38 (1H, m, 3′-H), 6.06 (1H, s, 8′-H), 6.11 (1H, dd-like, J 11 and 1, 10′-H), 6.25 (1H, br d, J 11.5, 14′-H), 6.33 (1H, d, J 15, 12′-H), 6.41 (1H, brd, J 11.5, 14-H), 6.55 (1H, dd, J 15 and 11, 11-H), 6.60 (1H, dd, J 15 and 11, 11′-H), 6.59 (1H, dd, J 14.5 and 11.5, 15-H), 6.65 (1H, d, J 15, 12-H), 6.73 (1H, dd, J 14.5 and 11.5, 15′-H) and 7.13 (1H, br d, J 11, 10-H).

### Example 4

### Synthesis of 4 (fucoxanthin succinate-4-vitamin C ester)

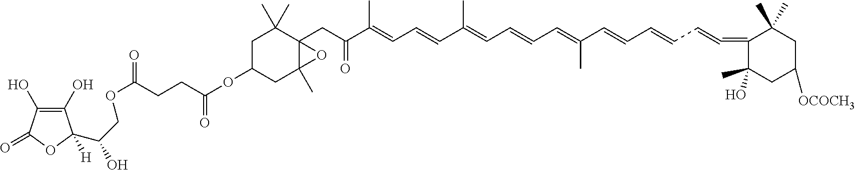

30 mmol fucoxanthin succinate is dissolved into 300 mL methylene chloride, 60 mmol 4-dimethylaminepyridine (DMAP), 45 mmol 2-O-tert-butyldimethylsilyl-ascorbic acid and 60 mmol N,N′-dicyclohexyl carbodiimide (DCC) were subsequently added. After 6-8 h, the reactant is vacuum filtrated to remove unreacted DCC and the ureide the reaction produced. After removing the solvent methylene chloride. The reactant mixture is chromatographically separate using a silica column chromatography to obtain a mahogany solid. The product is dried in vacuo, (yield: 30%). Subsequently, 0.06 mmol of the mahogany solid was dissolved in 6 mL tetrahydrofuran, stirred to uniformity, 0.06 mmol HF.Et₃N. was slowly added and the mixture was cooled to a temperature lower than 2° C. with continuation of stirring for 50 min. The mixture was then let stand still to warm to room temperature. Stirring of the reaction mixture was resumed for 2 h, then the reactant was poured into a separating funnel containing 10 mL ethyl acetate and 10 mL water to terminate the reaction. The organic layer was extracted twice with 10 mL water, dried in vacuo at a low temperature. After column chromatographic separation and condensation in vacuo at a low temperature, a mahogany solid, fucoxanthin succinate 4-vitamin C ester, was obtained.

ESI m/z: 903.45 (M⁺+1); (Found: M⁺, 902.45, C₅₁H₆₆O₁₄).

¹H NMR Data: δ_{H} (500 M Hz) 0.96 (3H, s, 1-Me^{eq}), 1.03 (3H, s, 1-Me^{ax}), 1.06 (3 H, s, 1′-Me^{eq}), 1.23 (3H, s, 5-Me), ˜1.36 (2-H^{ax}), 1.35 (3H, s, 5′-Me), 1.40 (3H, s, 1′-Me^{ax}), 1.42 (1H, t, J 12, 2′-H^{ax}), ˜1.48 (2-H^{eq}), 1.51 (1H, t, J 13, 4′-H^{ax}), 1.80 (1H, dd, J 14 and 9, 4-H^{ax}), 1.81 (3H, s, 9′-Me), 1.95 (3H, s, 9-Me), 1.98 (6H, s, 13-+13′-Me), ˜2.00 (2′-H^{eq}), 2.04 (3H, s, OAc), 2.28 (1H, ddd, J 13, 4 and 2, 4′-H^{eq}), 2.31 (1H, brdd, J 14 and 4.5, 4-H^{eq}), 2.60 and 3.66 (each 1H, d, J 18, 7-H₂), 2.70 (2H, dd, J 18, 3″-H₂), 2.65 (2H, dd, J 18, 2″-H₂), 5.32 (1H, m, 3-H), 5.38 (1H, m, 3′-H), 6.07 (1H, s, 8′-H), 6.11 (1H, dd-like, J 11 and 1, 10′-H), 6.26 (1H, br d, J 11.5, 14′-H), 6.34 (1H, d, J 15, 12′-H), 6.40 (1H, brd, J 11.5, 14-H), 6.56 (1H, dd, J 15 and 11, 11-H), 6.60 (1H, dd, J 15 and 11, 11′-H), 6.63 (1H, dd, J 14.5 and 11.5, 15-H), 6.66 (1H, d, J 15, 12-H), 6.74 (1H, dd, J 14.5 and 11.5, 15′-H) and 7.14 (1H, br d, J 11, 10-H).

### Example 5

### Synthesis of 5(fucoxanthin succinate 4-vitamin Cester sodium salt)

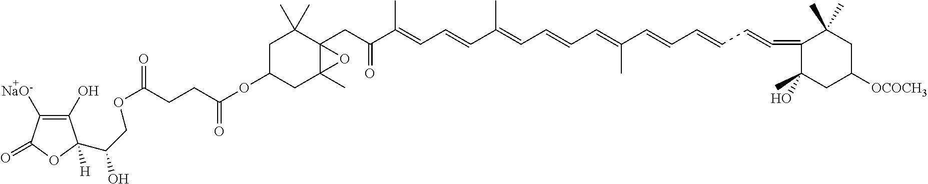

0.015 mmol fucoxanthin succinate 5-vitamin C ester was added into 10 mL tetrahydrofuran, the mixture was stirred until homogeneous, 0.075 mmol triethyl orthoformate was added with stirring, which was continued for stirring about 20 min, 0.0074 mmol sodium 2-ethylhexanoate solution in tetrahydrofuran was subsequently added dropwise into the reactant. The precipitation was filtered out, the filtrate was cooled to below 2° C., and added dropwise into 0.030 mmol sodium 2-ethylhexanoate solution in tetrahydrofuran. The reaction mixture was stirred for 10 min. After evaporation of the solvent, the residue was crystallized in acetone at a low temperature. The crystals was dried in vacuo to obtain fuscous solid. Yield: 40%.

MS: +APCI, m/z=904.43 (M+3H-Na⁺); (Found: M⁺, 924.43, C₅₁H₆₅NaO₁₄).

### Example 6

### Synthesis of 6(fucoxanthin citrate sodium salt)

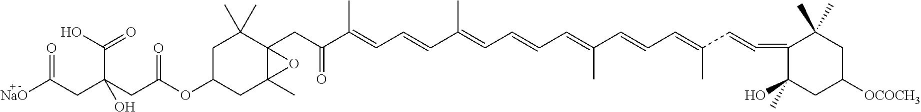

1 mmol fucoxanthin citrate was dissolved with stirring into 100 mL ethyl alcohol in a round bottom flask at room temperature and in an inert gas atmosphere 1.2 mmol solid sodium methylate was added, the mixture was stirred overnight. The precipitation was filtered out the next day, washed using small amount of ethyl alcohol, and subsequently with chloroform or methylene chloride to obtain a mahogany solid. After column chromatographic separation and repeated solvent recrystallization, fucoxanthin citrate sodium salt was obtained.

+APCI, m/z 835=(M+3H-Na⁺); (Found: M⁺, 855.43, C₄₈H₆₃NaO₁₂).

### Example 7

### Synthesis of 7(fucoxanthincitrate5-vitamin C ester)

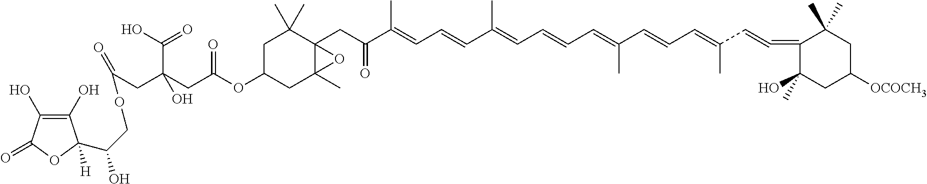

30 mmol fucoxanthin citrate was dissolved in 300 mL methylene chloride, 60 mmol 4-dimethylaminepyridine (DMAP), 45 mmol 2-O-tert-butyldimethylsilyl ascorbic acid and 60 mmol 1,3-dicyclohexylcarbodiimide (DCC) were added into the solution. After 6-8 h, the reactant is vacuum filtrated to remove unreacted DCC and the ureide the reaction produced. After removing the solvent methylene chloride, the reaction mixture was chromatographically separated by using a silica column. The separated eluent was condensed in vacuo at a low temperature to obtain a mahogany solid, the solid is dried in vacuo, yield: 10%.

0.06 mmol of this mahogany solid was dissolved into 6 mL tetrahydrofuran, the solution was stirred to uniformity, 0.06 mmol HF.Et₃N. was slowly added and the mixture was cooled to a temperature lower than 2° C. with incessant stirring for 50 min. The mixture was then let stand still to warm to room temperature. Stirring of the reaction mixture was resumed for 2 h, then the reactant was poured into a separating funnel containing 10 mL ethyl acetate and 10 mL water to terminate the reaction. The mixture was extracted twice with 10 mL water, separating to obtain the organic layer. The latter was dried in vacuo at a low temperature. After column chromatographic separation and purification, fucoxanthin citrate 5-vitaminester was obtained.

ESI m/z: 991 (M⁺+1); (Found: M⁺, 990.46, C₅₄H₇₀O₁₇).

¹HNMR Data: δ_{H} (500 M Hz) 0.95 (3H, s, 1-Me^{eq}), 1.02 (3H, s, 1-Me^{ax}), 1.05 (3 H, s, 1′-Me^{eq}), 1.22 (3H, s, 5-Me), ˜1.35 (2-H^{ax}), 1.34 (3H, s, 5′-Me), 1.39 (3H, s, 1′-Me^{ax}), 1.41 (1H, t, J 12, 2′-H^{ax}), 1.47 (2-H^{eq}), 1.50 (1H, t, J 13, 4′-H^{ax}), 1.81 (1H, dd, J 14 and 9, 4-H^{ax}), 1.80 (3H, s, 9′-Me), 1.94 (3H, s, 9-Me), 1.97 (6H, s, 13-+13′-Me), ˜2.00 (2′-H^{eq}), 2.03 (3H, s, OAc), 2.27 (1H, ddd, J 13, 4 and 2, 4′-H^{eq}), 2.30 (1H, brdd, J 14 and 4.5, 4-H^{eq}), 2.61 and 3.67 (each 1H, d, J 18, 7-H₂), 2.76 (2H, dd, J 18, 2″, 4″-H₂), 2.51 (2H, dd, J 18, 2″,4″-H₂), 5.30 (1H, m, 3-H), 4.36, 4.11 (2H, dd, J 18, 7, 6′), 4.44 (1H, m, 5′″), 5.0 (1H, 4″-H), 5.39 (1H, m, 3′-H), 6.06 (1H, s, 8′-H), 6.10 (1H, dd-like, J 11 and 1, 10′-H), 6.25 (1H, br d, J 11.5, 14′-H), 6.33 (1H, d, J 15, 12′-H), 6.39 (1H, brd, J 11.5, 14-H), 6.55 (1H, dd, J 15 and 11, 11-H), 6.59 (1H, dd, J 15 and 11, 11′-H), 6.62 (1H, dd, J 14.5 and 11.5, 15-H), 6.65 (1H, d, J 15, 12-H), 6.73 (1H, dd, J 14.5 and 11.5, 15′-H) and 7.13 (1H, br d, J 11, 10-H).

### Example 8

### Synthesis of 8 (fucoxanthin aconitate)

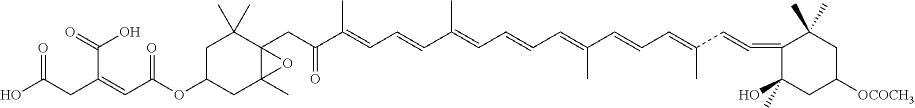

2 mmol fucoxanthin was dissolved in dry methylene chloride/tetrahydrofuran (95 mL/45 mL) at room temperature, the solution is added with 60 mmol N,N-Diisopropylethylamine, 20 mmol cis-aconitic anhydride, and 38 mmol 4-dimethylaminepyridine. The reaction mixture was stirred for 40 h at room temperature, subsequently diluted with methylene chloride. The reaction was terminated using NaCl/0.08M hydrochloric acid (25 mL/2 mL), and subsequently extracted with methylene chloride, the organic layers were mixed. The mixed solution was dried with anhydrous Na₂SO₄, condensed to obtain fucoxanthin aconitate. Yield: 40%.

ESI m/z: 815.43 (M⁺+1); (Found: M⁺, 814.43, C₄₈H₆₂O₁₁).

¹H NMR Data: δ_{H} (500 M Hz), 0.94 (3H, s, 1-Me^{eq}), 1.03 (3H, s, 1-Me^{ax}), 1.06 (3 H, s, 1′-Me^{eq}), 1.21 (3H, s, 5-Me), ˜1.34 (2-H^{ax}), 1.35 (3H, s, 5′-Me), 1.38 (3H, s, 1′-Me^{ax}), 1.40 (1H, t, J 12, 2′-H^{ax}), 1.46 (2-H^{eq}), 1.51 (1H, t, J 13, 4′-H^{ax}), 1.80 (1H, dd, J 14 and 9, 4-H^{ax}), 1.79 (3H, s, 9′-Me), 1.93 (3H, s, 9-Me), 1.96 (6H, s, 13-+13′-Me), ˜2.00 (2′-H^{eq}), 2.03 (3H, s, OAc), 2.26 (1H, ddd, J 13, 4 and 2, 4′-H^{eq}), 2.29 (1H, brdd, J 14 and 4.5, 4-H^{eq}), 2.60 and 3.66 (each 1H, d, J 18, 7-H₂), 6.27 (H, s, 2″-H), 2.92 (2H, dd, J 18, 4″-H₂), 5.32 (1H, m, 3-H), 5.38 (1H, m, 3′-H), 6.05 (1H, s, 8′-H), 6.09 (1H, dd-like, J 11 and 1, 10′-H), 6.24 (1H, br d, J 11.5, 14′-H), 6.32 (1H, d, J 15, 12′-H), 6.38 (1H, brd, J 11.5, 14-H), 6.54 (1H, dd, J 15 and 11, 11-H), 6.58 (1H, dd, J 15 and 11, 11′-H), 6.61 (1H, dd, J 14.5 and 11.5, 15-H), 6.64 (1H, d, J 15, 12-H), 6.72 (1H, dd, J 14.5 and 11.5, 15′-H) and 7.12 (1H, br d, J 11, 10-H).

### Example 9

### Synthesis of 9 (fucoxanthin di-methylaminobutyrate)

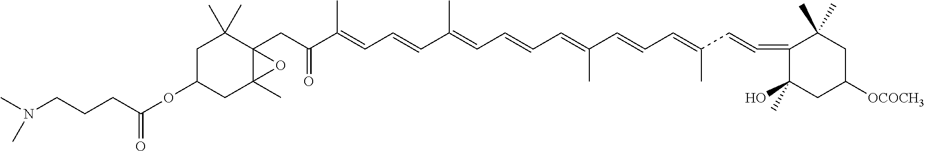

15 mmol 4-dimethylaminobutyric acid hydrochloride was added into the mixture of 100 mL methylene chloride and dimethylformamide (40:60) at room temperature, mixed into uniformity, 50 mmol N,N-Diisopropylethylamine, 150 mmol fucoxanthin and 450 mmol pyridine were subsequently added. The reaction mixture was stirred at room temperature for 40 h, The reaction mixture was then diluted with methylene chloride, The reaction was terminated with NaCl solution/0.08M hydrochloric acid (25 mL/2 mL), and subsequently extracted with methylene chloride, the organic layers were mixed. The mixed solution was desiccated with anhydrous Na₂SO₄, condensed to obtain fucoxanthin dimethylaminobutyrate. The product was purified by column chromatography. Yield: 50%.

ESI m/z (relative intensity): 772.51 (M⁺+1) (100), 771.51 (M⁺) (53%), (Found: M⁺, 771.51, C₄₈H₆₉NO₇)

¹HNMR Data: δ_{H} (500 M Hz) 0.95 (3H, s, 1-Me^{eq}), 1.03 (3H, s, 1-Me^{ax}), 1.05 (3 H, s, 1′-Me^{eq}), 1.20 (3H, s, 5-Me), ˜1.35 (2-H^{ax}), 1.36 (3H, s, 5′-Me), 1.37 (3H, s, 1′-Me^{ax}), 1.41 (1H, t, J 12, 2′-H^{ax}), ˜1.45 (2-H^{eq}), 1.50 (1H, t, J 13, 4′-H^{ax}), 1.79 (1H, dd, J 14 and 9, 4-H^{ax}), 1.80 (3H, s, 9′-Me), 1.92 (3H, s, 9-Me), 1.95 (6H, s, 13-+13′-Me), ˜2.00 (2′-H^{eq}), 2.02 (3H, s, OAc), 2.25 (1H, ddd, J 13, 4 and 2, 4′-H^{eq}), 2.28 (1H, brdd, J 14 and 4.5, 4-H^{eq}), 2.59 and 3.65 (each 1H, d, J 18, 7-H₂), 6.27 (2H, dd, 2″-H₂), 1.78 (2H, m, 3″-H₂), 2.36 (2H, dd, J7, 4″-H₂), 2.27 (6H, s, N,N-Me₂), 5.32 (1H, m, 3-H), 5.37 (1H, m, 3′-H), 6.04 (1H, s, 8′-H), 6.08 (1H, dd-like, J 11 and 1, 10′-H), 6.23 (1H, br d, J 11.5, 14′-H), 6.31 (1H, d, J 15, 12′-H), 6.37 (1H, brd, J 11.5, 14-H), 6.53 (1H, dd, J 15 and 11, 11-H), 6.57 (1H, dd, J 15 and 11, 11′-H), 6.60 (1H, dd, J 14.5 and 11.5, 15-H), 6.63 (1H, d, J 15, 12-H), 6.71 (1H, dd, J 14.5 and 11.5, 15′-H) and 7.11 (1H, br d, J 11, 10-H).

### Example 10

### Synthesis of 10 (fucoxanthin glutathione ester)

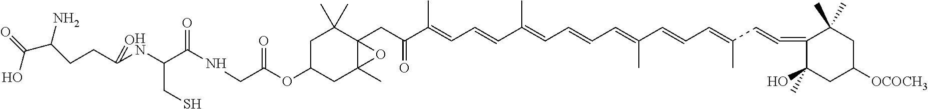

30 mmol fucoxanthin was dissolved into 300 mL methylene chloride, 60 mmol 4-dimethylaminepyridine (DMAP) and 60 mmol reduced glutathione were subsequently added, the mixture was cooled to 0° C., added with 60 mmol 1,3-dicyclohexylcarbodiimide (DCC). After 30 min, the ice bath was removed, the reactant was let stand still at room temperature for 2-4 h, was filtered to remove impurity dicyclohexyl urea, reacting for 6-8 h to obtain a mahogany solid fucoxanthin glutathione ester. The reactant mixture was purified using a silica column chromatography. Yield: 80%.

ESI m/z (relative intensity): M⁺947.5 (100%); (Found: M⁺, 947.50, C₅₂H₇₃N₃O₁₁S).

¹H NMR Data: δ_{H} (500 M Hz) 0.94 (3H, s, 1-Me^{eq}), 1.02 (3H, s, 1-Me^{ax}), 1.07 (3H, s, 1′-Me^{eq}), 1.21 (3H, s, 5-Me), ˜1.34 (2-H^{ax}), 1.35 (3H, s, 5′-Me), 1.37 (3H, s, 1′-Me^{ax}), 1.41 (1H, t, J 12, 2′-H^{ax}), ˜1.45 (2-H^{eq}), 1.50 (1H, t, J 13, 4′-H^{ax}), 1.79 (1H, dd, J 14 and 9, 4-H^{ax}), 1.78 (3H, s, 9′-Me), 1.92 (3H, s, 9-Me), 1.96 (6H, s, 13-+13′-Me), ˜2.00 (3H, m, 2′-H^{eq}, 11″-H), 2.02 (3H, s, OAc), 2.05 (2H, m, 9″-H), 2.25 (1H, ddd, J 13, 4 and 2, 4′-H^{eq}), 2.28 (1H, brdd, J 14 and 4.5, 4-H^{eq}), 2.61 and 3.65 (each 1H, d, J 18, 7-H₂), 3.18, 2.92 (2H, m, 12″-H₂), 3.48 (1H, dd, J 7, 7, 10″-H), 5.31 (1H, m, 3-H), 4.16, 1.5 (2H, d, J 18, 2″-H₂), 4.85 (1H, dd, J7,7, 5″-H), 5.39 (1H, m, 3′-H), 6.04 (1H, s, 8′-H), 6.08 (1H, dd-like, J 11 and 1, 10′-H), 6.23 (1H, br d, J 11.5, 14′-H), 6.31 (1H, d, J 15, 12′-H), 6.37 (1H, brd, J 11.5, 14-H), 6.53 (1H, dd, J 15 and 11, 11-H), 6.57 (1H, dd, J 15 and 11, 11′-H), 6.60 (1H, dd, J 14.5 and 11.5, 15-H), 6.63 (1H, d, J 15, 12-H), 6.71 (1H, dd, J 14.5 and 11.5, 15′-H) and 7.11 (1H, br d, J 11, 10-H), 8.0 (1H, s, 3″, 6″-H₂)

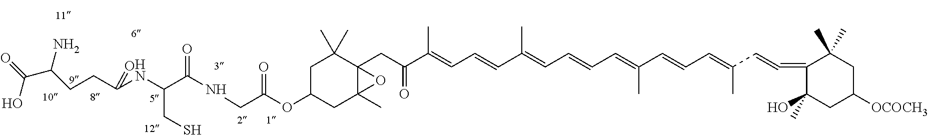

### Example 11

### Synthesis of 11 (fucoxanthin tartrate)

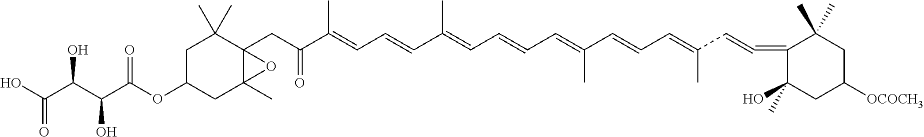

30 mmol fucoxanthin was dissolved in 300 mL methylene chloride/dioxane (10 mL/10 mL), added with 60 mmol 4-dimethylaminepyridine (DMAP) and 60 mmol (L)-tartaric acid, the mixture was cooled to 0° C., added with 60 mmol 1,3-dicyclohexylcarbodiimide (DCC). The ice bath was removed after 30 min. A mahogany solid was obtained after reacting for 6-8 h. The reaction mixture was chromatographed to obtain a mahogany solid, fucoxanthin tartrate. Yield: 80%.

ESI m/z (relative intensity): 791.43 (M⁺+1) (51.2%), 790.43 (M⁺) (100%), (Found: M⁺, 790.43, C₄₆H₆₂O₁₁).

¹H NMR Data: δ_{H} (500 M Hz) 0.94 (3H, s, 1-Me^{eq}), 1.04 (3H, s, 1-Me^{ax}), 1.06 (3 H, s, 1′-Me^{eq}), 1.21 (3H, s, 5-Me), ˜1.35 (2-H^{ax}), 1.37 (3H, s, 5′-Me), 1.36 (3H, s, 1′-Me^{ax}), 1.42 (1H, t, J 12, 2′-H^{ax}), ˜1.46 (2-H^{eq}), 1.51 (1H, t, J 13, 4′-H^{ax}), 1.80 (1H, dd, J 14 and 9, 4-H^{ax}), 1.81 (3H, s, 9′-Me), 1.93 (3H, s, 9-Me), 1.96 (6H, s, 13-+13′-Me), ˜2.00 (2′-H^{eq}), 2.01 (3H, s, OAc), 2.26 (1H, ddd, J 13, 4 and 2, 4′-H^{eq}), 2.29 (1H, brdd, J 14 and 4.5, 4-H^{eq}), 2.60 and 3.66 (each 1H, d, J 18, 7-H₂), 4.46 (H, d, J 7,2″-H), 4.81 (H, d, J 7,3″-H), 5.31 (1H, m, 3-H), 5.36 (1H, m, 3′-H), 6.03 (1H, s, 8′-H), 6.04 (1H, dd-like, J 11 and 1, 10′-H), 6.24 (1H, br d, J 11.5, 14′-H), 6.32 (1H, d, J 15, 12′-H), 6.38 (1H, brd, J 11.5, 14-H), 6.54 (1H, dd, J 15 and 11, 11-H), 6.58 (1H, dd, J 15 and 11, 11′-H), 6.61 (1H, dd, J 14.5 and 11.5, 15-H), 6.64 (1H, d, J 15, 12-H), 6.72 (1H, dd, J 14.5 and 11.5, 15′-H) and 7.12 (1H, br d, J 11, 10-H).

### Example 12

### Synthesis of 12 (fucoxanthol bi-morpholino carbamate)

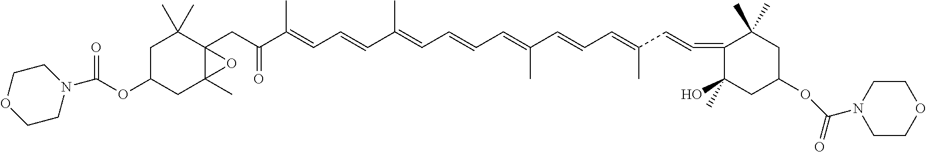

3.98 mmol N,N-Diisopropylethylamine, 2.7 mmoL4-dimethylaminopyridine and 1.35 mmol 4-morpholinocarboxyl chloride were dissolved into the solution of 0.135 mmol fucoxanthol in methylene chloride/dioxane (10 mL/6 mL) at room temperature. The reaction mixture was stirred at room temperature for 40 h, the reactant was then diluted with methylene chloride, cooled to below room temperature, and subsequently extracted with methylene chloride, the organic layers were pooled, the pooled solution desiccated with anhydrous Na₂SO₄, condensed to obtain fucoxanthol bimorpholino carbamate (Yield: 64%). The product was analyzed by TLC and HPLC.

ESI m/z (relative intensity): M⁺+1843.51 (53%), M⁺842.51 (100%); (Found: M⁺, 842.51, C₅₀H₇₀N₂O₉).

¹HNMR Data: δ_{H} (500 M Hz) 0.94 (3H, s, 1-Me^{eq}), 1.04 (3H, s, 1-Me^{ax}), 1.06 (3 H, s, 1′-Me^{eq}), 1.21 (3H, s, 5-Me), ˜1.35 (2-H^{ax}), 1.37 (3H, s, 5′-Me), 1.36 (3H, s, 1′-Me^{ax}), 1.42 (1H, t, J 12, 2′-H^{ax}), 1.46 (2-H^{eq}), 1.51 (1H, t, J 13, 4′-H^{ax}), 1.80 (1H, dd, J 14 and 9, 4-H^{ax}), 1.81 (3H, s, 9′-Me), 1.93 (3H, s, 9-Me), 1.96 (6H, s, 13-+13′-Me), ˜2.00 (2′-H^{eq}), 2.01 (3H, s, OAc), 2.26 (1H, ddd, J 13, 4 and 2, 4′-H^{eq}), 2.29 (1H, brdd, J 14 and 4.5, 4-H^{eq}), 2.60 and 3.66 (each 1H, d, J 18, 7-H₂), 3.67 (8H, m, 2″-H₂, 2′″-H₂), 3.47 (8H, m, 3″-H₂, 3′″-H₂), 5.32 (1H, m, 3-H), 5.38 (1H, m, 3′-H), 6.03 (1H, s, 8′-H), 6.04 (1H, dd-like, J 11 and 1, 10′-H), 6.24 (1H, br d, J 11.5, 14′-H), 6.32 (1H, d, J 15, 12′-H), 6.38 (1H, brd, J 11.5, 14-H), 6.54 (1H, dd, J 15 and 11, 11-H), 6.58 (1H, dd, J 15 and 11, 11′-H), 6.61 (1H, dd, J 14.5 and 11.5, 15-H), 6.64 (1H, d, J 15, 12-H), 6.72 (1H, dd, J 14.5 and 11.5, 15′-H) and 7.12 (1H, br d, J 11, 10-H).

### Example 13

### Synthesis of 13 (fucoxantholmannitol carbonate)

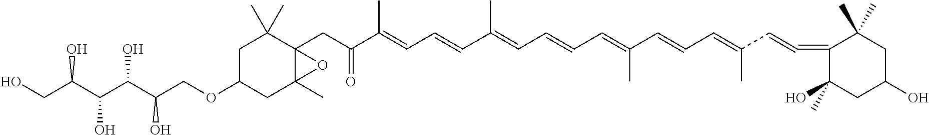

N,N-Diisopropylethylamine and 1,2,2,2-tetrafluoroethyl chloroformate were added into the solution of 0.1 mmol fucoxanthol in 45 mL methylene chlorideat below room temperature. After being stirred, 0.2 mmol(D)-mannitol, 10 mL dimethylformamide and 2.0 mmol 4-dimethylaminopyridine were added into the reactant. The reaction mixture was stirred for 20 h at room temperature, after which the reactant was diluted using methylene chloride, the reaction was terminated using salt solution, and subsequently extracted with methylene chloride. The organic layers were pooled, the pooled solution condensed to obtain fucoxantholmannitol carbonate. (Yield: 46%): The product was analyzed by TLC and HPLC.

ESI m/z (relative intensity): 811.46 (M⁺+1) (51.0%), 810.46 (M⁺) (100%), (Found: M⁺,810.46, C₄₆H₆₆O₁₂).

¹HNMR Data: δ_{H} (500 M Hz) 0.95 (3H, s, 1-Me^{eq}), 1.05 (3H, s, 1-Me^{ax}), 1.07 (3H, s, 1′-Me^{eq}), 1.22 (3H, s, 5-Me), ˜1.34 (2-H^{ax}), 1.37 (3H, s, 5′-Me), 1.36 (3H, s, 1′-Me^{ax}), 1.42 (1H, t, J 12, 2′-H^{ax}), 1.46 (2-H^{eq}), 1.50 (1H, t, J 13, 4′-H^{ax}), 1.79 (1H, dd, J 14 and 9, 4-H^{ax}), 1.82 (3H, s, 9′-Me), 1.92 (3H, s, 9-Me), 1.95 (6H, s, 13-+13′-Me), ˜2.00 (2′-H^{eq}), 2.01 (3H, s, OAc), 2.25 (1H, ddd, J 13, 4 and 2, 4′-H^{eq}), 2.28 (1H, brdd, J 14 and 4.5, 4-H^{eq}), 2.59 and 3.66 (each 1H, d, J 18, 7-H₂), 6.0 (1H, d, J_{7,1}″-H), 3.97 (1H, dd, J7, 7, 2″-H), 3.37 (1H, dd, J7, 7, 3″-H), 3.38 (1H, m, 4″-H), 3.81, 3.56 (2H, m, 5″-H₂), 5.30 (1H, m, 3-H), 4.32 (1H, m, 3′-H), 6.02 (1H, s, 8′-H), 6.05 (1H, dd-like, J 11 and 1, 10′-H), 6.25 (1H, br d, J 11.5, 14′-H), 6.32 (1H, d, J 15, 12′-H), 6.38 (1H, brd, J 11.5, 14-H), 6.53 (1H, dd, J 15 and 11, 11-H), 6.58 (1H, dd, J 15 and 11, 11′-H), 6.60 (1H, dd, J 14.5 and 11.5, 15-H), 6.63 (1H, d, J 15, 12-H), 6.73 (1H, dd, J 14.5 and 11.5, 15′-H) and 7.13 (1H, br d, J 11, 10-H).

### Example 14

### Synthesis of 14 (fucoxanthin resveratrol succinate)

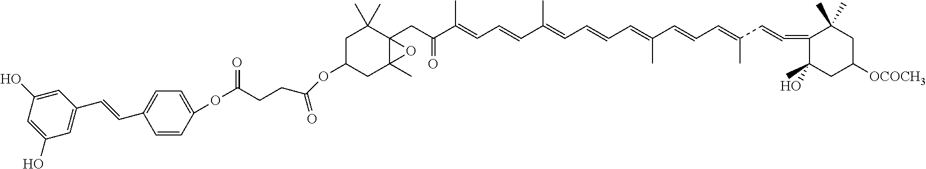

60 mmol resveratrol was dissolved into 300 mL methylene chloride/dioxane (10 mL/10 mL), 60 mmol 4-dimethylaminepyridine (DMAP) and 30 mmol fucoxanthin succinate were subsequently added. The mixture was cooled to 0° C., added with 60 mmol 1,3-dicyclohexylcarbodiimide (DCC), 30 min later the ice bath was removed. After reaction at room temperature for 6-8 h, the reactant is vacuum filtrated to remove unreacted DCC and the ureide the reaction produced. After removing the solvent methylene chloride, the reaction mixture was chromatographically separated by using a silica column chromatography. The separated eluent was condensed in vacuo at a low temperature to obtain fucoxanthin resveratrol succinate.

ESI m/z (relative intensity): 969.51 (M⁺+1) (66.1%), 968.51 (M⁺+1); (Found: M⁺, 968.51, C₆₀H₇₂O₁₁, theoretical M: 968.51.).

### Example 15

### Synthesis of 15 (fucoxanthin linoleate)

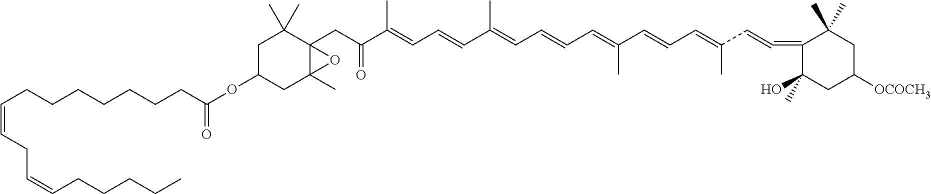

30 mmol fucoxanthin was dissolved in 300 mL methylene chloride/dioxane (10 mL/10 mL), 60 mmol 4-dimethylaminepyridine (DMAP) and 60 mmol (L)-linoleic acid were added subsequently, the mixture was cooled to 0° C., 60 mmol 1,3-dicyclohexylcarbodiimide (DCC) was added, 30 minutes later, the ice bath was removed. After reacting at room temperature for 6-8 h, the reactant is vacuum filtrated to remove unreacted DCC and the ureide the reaction produced. After removing the solvent methylene chloride, the reaction mixture was chromatographically separated by a silica column chromatography. The separated eluent was condensed in vacuo at a low temperature to obtain a mahogany solid fucoxanthin linoleate.

ESI m/z (relative intensity): 921.65 (M⁺+1); (Found: M⁺, 920.65, C₆₀H₆₈O₇).

### Example 16

### Synthesis of 16 (fucoxanthin monobenzyl ether)

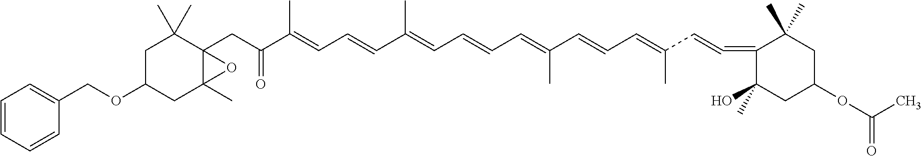

Potassium bis(trimethylsilyl)amide (1.5 mmol, in toluene) was added into a solution of fucoxanthin (0.1 mmol) and benzyl chloride (1.5 mmol) in methylene chloride/dimethyl sulfoxide (50 mL/50 mL) at a low temperature. The mixture was stirred at 0° C. for 65 min, let stand to warm to room temperature. The mixture was stirred at room temperature to complete the reaction, the reactant was diluted with methylene chloride, the reaction was terminated using salt solution/hydrochloric acid, subsequently extracted with methylene chloride, the methylene chloride layers were pooled and pooled solution was condensed, separated with column chromatography, and condensed in vacuo to obtain fucoxanthin monobenzyl ether.

ESI m/z (relative intensity): 749.47 (M⁺+1); (Found: M⁺, 748.47, C₄₉H₆₄O₆)

### Example 17

### Testing Compound: Any one of Compounds 1-16

Formulation of the feedstuff: high fat content feedstuff was composed of 10% lard, 80% basal feed and 10% yolk powder.

Grouping and treatment of the experimenting animals: healthy standard clean grade SD adult male rats, body weight 180˜210 g, were fed adaptively with basal feed for a week, 10 of the animals were used as basal feed control, the rest were administrated with high-fat feed. 1 month later, the rats administrated with high-fat feed were grouped randomly according to their body weights into model control group, fucoxanthin group, and test animal groups, each group had 10 rats. Fucoxanthin group and test animal groups were administrated with same doses of the drugs. The basal feed control group continued to be fed with basal feed, other groups with high-fat feed. The basal feed- and model control groups were intragastric administrated with distilled water, the other, drug-administrated groups with respective doses of drug, the administration were carried out uninterruptedly for 30 d. Each group of the animals were bred in different hutches, the room temperature was controlled within (22±2)° C., using natural illumination, the animals took food and drank freely. The body weights of the animals were measured one time each week, feed-intakes of the rats were also observed and recorded. After 30 days the rats were weighed.

Statistical analysis: Variance analysis of the data collected in this experiment was implemented by SAS software package, Dunnett's t-test was used for inter-group comparative analysis, p<0.05 was used as the criterion of statistical significance.

Results: according to the experimental records, food-intake of all groups of rats showed no difference along with the drug administrating time, there was no statistical significance. This none-significance will not be recounted later. From Table 1 it can be seen that compared with the basal feed control, there was statistical significance between body weights of model control- and drug-administrated animals after the completion of the experiment (p<0.05). This meant the success of the rat obesity modeling.

Meanwhile, the body weight increases of the drug-administrated animals were further reduced in comparison with the model control animals (p<0.05, p<0.01), this meant that, after the structural modification, fucoxanthin- and fucoxanthol derivatives showed stronger weight-reducing effects than their respective parent compounds. This will have certain instructive effect on clinic in the future.

TABLE 1
Impact of different drug-administrated groups on the body
weights of nutritional obesity rats ( ₓ̅ ± s) g

| | Body weight(g) | Body weight(g) | Body |
|---|---|---|---|
| | (before- | (After- | weight(g) |
| Group | experiment) | experiment) | increased) |
| basal feed control | 438.21 ± 21.20 | 469.43 ± 32.78 | 31.22 ± 19.87 |
| model control | 485.98 ± 20.45* | 547.28 ± 35.52 | 61.30 ± 21.22* |
| fucoxanthin | 486.50 ± 22.10 | 531.00 ± 20.32Δ | 43.68 ± 19.13Δ |
| fucoxanthol | 487.20 ± 21.20 | 527.00 ± 18.89ΔΔ | 39.39 ± 18.11ΔΔ |
| compound 12 | 487.13 ± 19.18 | 500.01 ± 20.10ΔΔ | 12.12 ± 1.25ΔΔ |
| compound 13 | 486.13 ± 20.00 | 502.11 ± 18.99ΔΔ | 15.23 ± 0.34ΔΔ |
| compound 7 | 487.25 ± 19.12 | 506.00 ± 19.07ΔΔ | 18.21 ± 1.12ΔΔ |
| compound 6 | 485.89 ± 18.99 | 507.00 ± 20.11ΔΔ | 20.32 ± 1.21ΔΔ |
| compound 1 | 486.12 ± 19.43 | 515.00 ± 29.21ΔΔ | 28.50 ± 1.15ΔΔ |
| compound 4 | 486.21 ± 20.11 | 514.00 ± 30.22ΔΔ | 27.41 ± 1.21ΔΔ |
| compound 14 | 485.15 ± 21.12 | 514.00 ± 22.22ΔΔ | 28.52 ± 1.21ΔΔ |
| compound 11 | 485.35 ± 20.10 | 515.00 ± 18.90ΔΔ | 29.25 ± 1.23ΔΔ |

(Note:
In comparison with the basal feed control group, *p < 0.05; in comparison with the model control group, Δp < 0.05, ΔΔp < 0.01)

### Example 18

Experimental procedures were the same as in Example 17. After feeding for 30 d, all the rats were executed after weighing, the fat pads around testicles were peeled off, weighed precisely. Statistical analysis was the same as in Example 16.

After the completion of the experiment, in comparison with the basal feed control group, there was statistical significance between body weights of the rats in model control groups as well as their weights of fat pads around the testicles/body weights (p<0.05), This meant the success of the rat obesity modeling.

Meanwhile, the body weights of the rats in groups administrated with drugs, weights of fat pads around their testicles, their weights of fat pads around the testicles/body weights were lower than the model control (p<0.05). This meant that fucoxanthin- or fucoxanthol derivatives showed weight-reducing effect on obese rats, more prominent than single dose of fucoxanthin or fucoxanthol.

TABLE 2
Impact of different drug-administrated groups on the weights of
fat around their testicles of nutritional obesity rats ( ₓ̅ ± s)g

| | Body weight(g) | Weight fat pads | Weight fat pads |
|---|---|---|---|
| | (After- | around | around |
| Group | experiment) | testicles(g) | testicles/bw × 100 |
| Basal feed control | 469.43 ± 32.78 | 6.44 ± 0.88 | 1.37 ± 0.15 |
| Model control | 547.28 ± 35.52* | 9.21 ± 0.51* | 1.68 ± 0.14* |
| fucoxanthin | 531.00 ± 20.32 | 7.32 ± 0.83Δ | 1.38 ± 0.11Δ |
| fucoxanthol | 527.00 ± 18.89 | 7.00 ± 0.67ΔΔ | 1.25 ± 0.13ΔΔ |
| compound 13 | 515.01 ± 18.89 | 3.00 ± 0.12ΔΔ | 0.60 ± 0.11ΔΔ |
| compound 4 | 514.00 ± 30.22 | 4.00 ± 0.14ΔΔ | 0.70 ± 0.21ΔΔ |
| compound 14 | 514.00 ± 22.22 | 5.00 ± 0.12ΔΔ | 0.90 ± 0.12ΔΔ |
| compound 2 | 512.1 ± 21.00 | 6.00 ± 0.11ΔΔ | 1.18 ± 0.13ΔΔ |
| compound 15 | 520.01 ± 16.88 | 6.34 ± 0.55ΔΔ | 1.22 ± 0.11ΔΔ |
| Compound 3 | 513.1 ± 19.13 | 7.00 ± 0.13ΔΔ | 1.20 ± 0.10ΔΔ |

(Note:
In comparison with the basal feed control group, *p < 0.05; in comparison with the model control group, Δp < 0.05, ΔΔp < 0.01)

### Example 19

Experimental procedures were the same as in Example 17. After feeding for 30 d, all the rats were executed after weighing, the fat pads around kidneys were peeled off, weighed precisely. Statistical analysis was the same as in Example 16.

In comparison with the basal feed control group, there was statistical significance between body weights of the rats in model control groups, weights of the fat pads around their kidneys, as well as their weights of fat pads around kidney/body weights (p<0.05), This meant the success of the rat obesity modeling.

Meanwhile, body weights of the rats in groups administrated with drugs, the weights of the fat pads around their kidneys, as well as weights of fat pads around their kidneys/body weights were lower than those of the model control, this meant that the groups fed with fucoxanthin and fucoxanthol derivatives showed very significant weight reduction effect, more significant than fucoxanthin and fucoxanthol.

TABLE 3
Impact of different drug-administrated groups on the weights of
fat around the kidneys of nutritional obesity model rats ( ₓ̅ ± s) g

| | Body weight(g) | Weight fat | Weight fat pads |
|---|---|---|---|
| | (After- | pads around | around |
| Group | experiment) | kidneys(g) | kidneys/bw × 100 |
| basal feed control | 469.43 ± 32.78 | 5.57 ± 0.46 | 1.19 ± 0.12 |
| model control | 547.28 ± 35.52* | 9.97 ± 0.65* | 1.82 ± 0.13* |
| fucoxanthin | 531.00 ± 20.32 | 7.42 ± 0.60Δ | 1.42 ± 0.12Δ |
| fucoxanthol | 527.00 ± 18.89 | 7.0 ± 0.58ΔΔ | 1.20 ± 0.10ΔΔ |
| compound 10 | 522.08 ± 18.11 | 4.00 ± 0.40ΔΔ | 0.75 ± 0.11ΔΔ |
| compound 8 | 518.18 ± 15.21 | 4.40 ± 0.34ΔΔ | 0.85 ± 0.12ΔΔ |
| compound 9 | 514.13 ± 13.00 | 5.00 ± 0.23ΔΔ | 0.98 ± 0.11ΔΔ |
| compound 5 | 513.15 ± 11.00 | 7.00 ± 0.32ΔΔ | 1.12 ± 0.10ΔΔ |

(Note:
In comparison with the basal feed control group, *p < 0.05; in comparison with the model control group, Δp < 0.05, ΔΔp < 0.01)

### Example 20

Experimental procedures were the same as in Example 17. After feeding for 30 d, all the rats were executed after weighing, their abdomen fat were peeled off, weighed precisely. Statistical analysis was the same as in Example 16.

Results: in comparison with the basal feed control group, there was statistical significance between body weights of the rats in model control groups, their abdomen fat weights as well as their abdomen fat weights/body weights (p<0.05), This meant the success of the rat obesity modeling.

The body weights of the rats in respective groups weights of their abdomen fat, weights of their abdomen fat/body weights were further reduced (p<0.05). This meant that groups fed with fucoxanthin esterified derivatives showed weight-reducing effect on obese rats, more prominent than single dose of fucoxanthin.

TABLE 4
Impact of different drug-administrated groups on the weights
of abdomen fat of nutritional obesity rats ( ₓ̅ ± s) g

| | Body weight(g) | Weight. | Weight |
|---|---|---|---|
| | (After- | abdomenfat | abdomen |
| Group | experiment) | (g) | fat/bw × 100 |
| basal feed control | 469.43 ± 32.78 | 8.98 ± 2.04 | 1.92 ± 0.11 |
| Model control | 547.28 ± 35.52 | 13.29 ± 1.23* | 2.43 ± 0.22* |
| fucoxanthin | 531.00 ± 20.32 | 9.61 ± 0.12ΔΔ | 1.81 ± 0.13Δ |
| fucoxanthol | 527.00 ± 18.89 | 7.91 ± 0.11ΔΔ | 1.60 ± 0.11ΔΔ |
| compound 10 | 517.00 ± 11.23 | 7.75 ± 0.20ΔΔ | 1.50 ± 0.14ΔΔ |
| compound 11 | 515.00 ± 18.90 | 7.21 ± 0.23ΔΔ | 1.40 ± 0.13ΔΔ |
| compound 2 | 516.03 ± 12.15 | 6.76 ± 0.22ΔΔ | 1.31 ± 0.12ΔΔ |
| compound 4 | 514.00 ± 30.22 | 4.11 ± 0.14ΔΔ | 0.80 ± 0.11ΔΔ |
| compound 5 | 504.01 ± 18.23 | 3.58 ± 0.21ΔΔ | 0.71 ± 0.10ΔΔ |
| compound 13 | 502.11 ± 18.99 | 3.11 ± 0.24ΔΔ | 0.62 ± 0.12ΔΔ |

(Note:
In comparison with the basal feed control group *p < 0.05; in comparison with the model control group, Δp < 0.05, ΔΔp < 0.01)

Above are only the preferable embodiment of this invention. The only serve to illustrate, but not restrict the scope of this invention. The technicians of this specialty understand that many changes, modifications and even conventional substitution can be imposed on this invention within the spirit and scope of this invention claims, however, they will all fall into the protection range of this invention.

What is claimed is:

1. A compound having weight reducing effect, the compound represented by Formula I or its medicinally acceptable salt thereof: 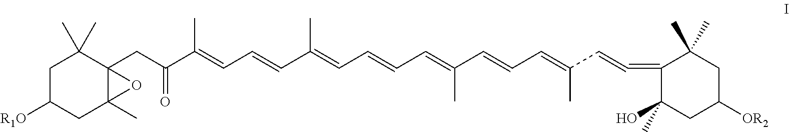
wherein R₁ and R₂ are independently selected from the group consisting of hydrogen, acetyl, citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, morpholinocarbamoyl, mannitol carbonyl, hexadecanoyl, linoleyl, linolenyl, arachidonyl, a group forming ether with the oxygen conjugating with R₁ or R₂, esterified citryl, and esterified succinyl; and
R₁ and R₂ are not hydrogen at the same time.

2. The compound or their medicinally acceptable salt of claim 1, wherein when said R₁ is citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, hexadecanoyl, linoleyl, linolenyl, arachidonyl, the group individually forming ether with the oxygen conjugating with R₁ or R₂, or esterified citryl or esterified succinyl, said R₂ is acetyl.

3. The compound of claim 1 or 2, wherein said group forming ether with the oxygen conjugating with R₁ or R₂ is C₁₋₆ alkyl, C₆₋₁₂ aryl, aryl C₁₋₄ alkyl, C₁₋₉heteroaryl or C₁₋₄ alkylheteroaryl.

4. The compound of claim 3, wherein said group forming ether with the oxygen conjugating with R₁ or R₂ is aryl C₁₋₄alkyl.

5. The compound of claim 4, wherein said group forming ether with the oxygen conjugating with R₁ or R₂ is benzyl.

6. The compound of claim 1 or 2, wherein said esterified citryl or esterified succinyl include at least one of the following structures:

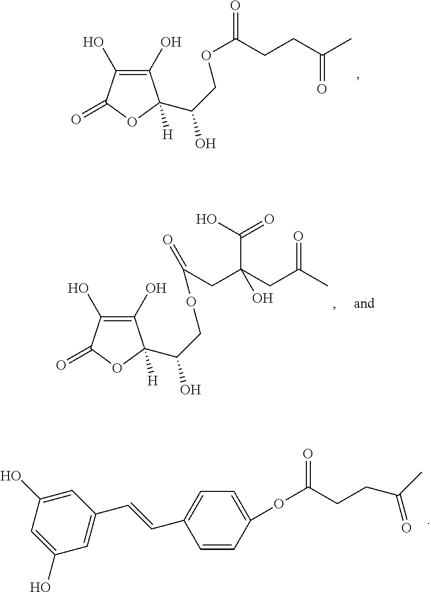

7. The compound of claim 1 or 2, wherein said medicinally acceptable salt thereof is sodium or potassium salt, formed by a reaction of the compound of general formula I with alkaline metal.

8. The compound of claim 1 or 2, wherein R₁ is citryl or citryl sodium salt, succinyl or succinyl sodium salt, or 1-substituted-ascorbic acid succinyl, 1-substituted-ascorbic acid citryl or its sodium salt, and R₂ is acetyl.

9. A drug composition, comprising: medicinally acceptable adjuvant or diluent and the compound of claim 1.

10. A method to prepare the compound of claim 1, comprising one of the following steps:
(i) wherein fucoxanthin is the raw material: 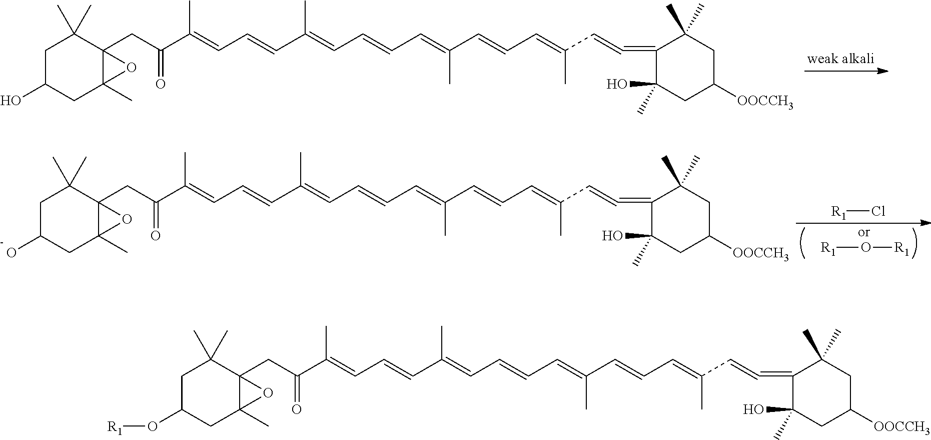
;
(ii) wherein fucoxanthol is the raw material: 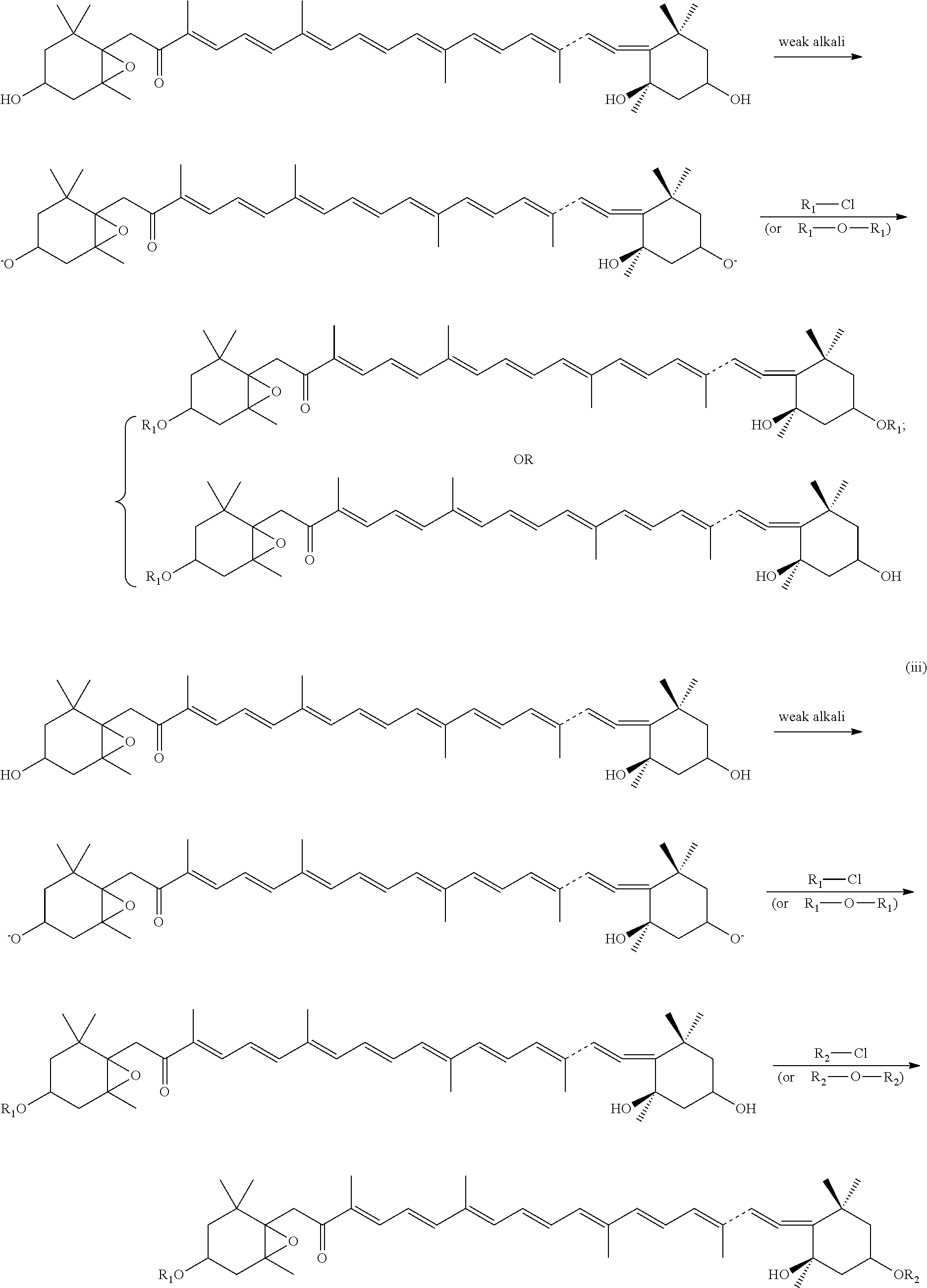
or
wherein R₁ and R₂ are independently selected from the group consisting of hydrogen, acetyl, citryl, succinyl, aminoacyl, 1-substituted-ascorbic acid succinyl, 1-substituted-ascorbic acid citryl, dimethylphosphoryl, aconityl, dimethylaminobutyryl, glutathionyl, tartaryl, morpholinocarbamoyl, mannitol carbonyl, hexadecanoyl, linoleyl, linolenyl, arachidonyl, a group forming ether with the oxygen conjugating with R₁ or R₂, esterified citryl, and esterified succinyl; and
R₁ and R₂ are not hydrogen at the same time.

11. The preparation method of claim 10, wherein the esterification s performed by the following DCC method: 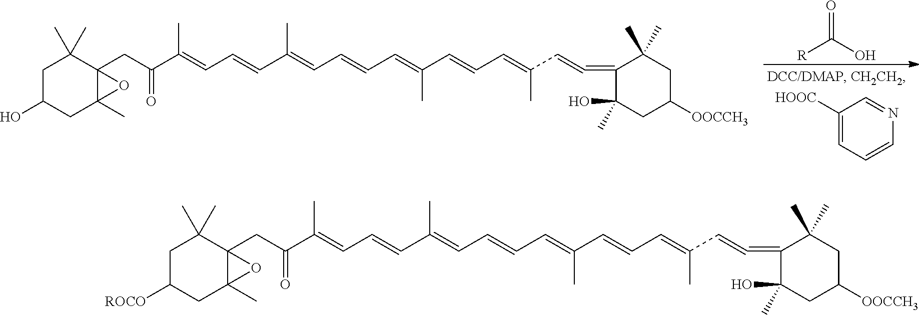
wherein R is individually alkyl, or substituted-alkyl.

12. The preparation method of claim 10, wherein the fucoxanthin or fucoxanthol is from a plant, a microorganism, an animal, or from a synthetic way.

13. A method of reducing weight, comprising:
administering a subject with at least one of said compound of claim 1, their medicinal acceptable salt, and drug composition, wherein said subject is mammal.

14. The method of claim 13, wherein said weight reducing is body weight reduction of the subject.

15. The method of claim 13, wherein said weight reduction is the reduction of abdomen fat of the subject.

16. The method of claim 13, wherein the administration of the compound or drug composition of claim 1 to the subject comprises oral, topical, intravenous, intramuscular or subcutaneous administration of the compound or drug composition to the subject.
